(19) United States
(12) Patent Application Publication
(10) Pub. No.: US 2005/0003017 A1
(43) Pub. Date: Jan. 6, 2005

(54) Hypotensive and hypertensive properties of renomedullar extracts

(76) Inventor: Bernhard Glodny, Munster (DE)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/296,067

(22) PCT Filed: May 22, 2001
(86) PCT No.: PCT/EP01/05872

(30) Foreign Application Priority Data
May 26, 2000 (EP) 00111350.5

(51) Int. Cl. A61K 35/23

(52) U.S. Cl. 424/558

(57) ABSTRACT

Hypotensive and Hypertensive Activities from the Renal Medulla The present invention relates to novel hypotensive and hypertensive extracts obtainable from the renal medulla, methods for the isolation thereof from the renal medulla of mammals, pharmaceutical compositions comprising the respective extracts, and their use for lowering and raising the blood pressure in mammals, especially humans.

The present invention relates to novel hypotensive and hypertensive extracts obtainable from the renal medulla, methods for the isolation thereof from the renal medulla of mammals, pharmaceutical compositions comprising the respective extracts, and their use for lowering and raising the blood pressure in mammals, especially humans.

### BACKGROUND OF THE INVENTION

The kidney contains a number of substances having an effect on the blood pressure. These substances can be classified into hypotensive substances (lowering the blood pressure), such as prostaglandin E2 or prostaglandin A2, its artificial aglycon, and hypertensive substances (raising the blood pressure), such as thromboxane A2. Most of these substances, such as nephrotensin, renotensin, corticopressin or medullipin, have not yet been isolated in a pure form, and their structures are still unknown; Thromboxane A2 has not been isolated in a pure form from the kidney either. Therefore, great attention was roused in 1941 when Page et al. (Page I. H., Helmer O. M., Kohlstaedt K. G., Gambill W. D., Taylor R. D. in “The blood pressure reducing property of extracts of kidneys in hypertensive patients and animals” Ann. Int. Med. 15, 347 (1941)) reported that changes of the eye-ground in patients afflicted with malignant hypertension could be made to retrograde and hypertension reduced by means of kidney extracts. With experiments made on dogs in which renoprival hypertension could be alleviated by intraperitoneal administration of renal medulla, but not of renal cortex (Muirhead E. E., Jones F., Stirman J. A. in “Antihypertensive property in renoprival hypertension of extracts of renal medulla” J. Lab. Clin. Med. 56, 167 (1960)), it became clear that the hypotensive principle must be contained in the renal medulla. This eventually resulted in the isolation of the strongly vasodilatory prostaglandins A2 and E2 from renal medulla (Daniels E. G., Hinman J. W., Leach B. E., Muirhead E. E. in “Identification of prostaglandin E2 as the principal vasodepressor lipid of rabbit renal medulla” Nature 215, 1298 (1967)). Prostaglandin E2 was described as the mainly effective hypotensive principle of the renal medulla (Daniels, supra).

The antihypertensive neutral renomedullary lipid (ANRL), which was later renamed medullipin (Muirhead E. E. in “Renomedullary Vasodepressor Lipid: Medullipin” in “Textbook of hypertension”, editor: J. D. Swales. Oxford, Blackwell, 1994, referred to as “textbook” in the following), could not be purified to date (Muirhead E. E. in “textbook”, supra).

The material-chemical assignment of various observed physiological phenomena to a molecule “medullipin” (Muirhead E. E. in “textbook”, supra) is still hypothetical. Thus, to date, there has been only the chemically unascertained assumption that medullipin had two vicinal hydroxy groups (Muirhead E. E., Byers L. W., Brooks B., Brown P., Pitcock J. A. in “Biologic contrasts between Medullipin I and vasoactive glyceryl compounds”, Am. J. Med. Sci. 1989; 298: 93-103). To date, medullipin has been characterized only in physiological terms:
With respect to occurrence in mammals: Medullipin is contained in the renal medulla of humans, rabbits and rats (Muirhead E. E. in “textbook”, supra).
With respect to recovery: It can be recovered from plasma from the vein of a kidney which is perfused with an increased perfusion pressure. Such plasma has a hypotensive effect on spontaneously hypertensive rats which is directly dependent on the perfusion pressure. In addition, it can be recovered from fresh renal medulla, as well as from the effluate of declipped Goldblatt kidneys, and from supernatants and extracts of interstitial renal medullar cells (Muirhead E. E. in “textbook”, supra).

With respect to physiological properties: It reduces hypertension, with a delay, within a few minutes. This effect is maintained for several hours (Muirhead E. E. in “Depressor Functions of the Kidney”, Seminars in Nephrology, Vol. 3, No. 1 (March), 14-29 (1983)). After removal of the liver from the circulation and subsequent administration of medullipin, medullipin has no longer any vasodepressor properties (Muirhead E. E. in “textbook”, supra). Also, the activity is lost upon previous administration of proadifen (SKF525A) (Muirhead E. E. in “textbook”, supra).

Works in which hypertensive mixtures or substances are described include:
1.) The earliest work (Tigerstedt R., Bergmann P. G. in “Niere und Kreislauf”, Skand. Arch. Physiol. 8, 223 (1898)), which resulted in the isolation of renin, which itself is not vasoeffective (Haas E., Lamfrom H., Goldblatt H. in “Isolation and purification of hog renin” Arch. Biochem. Biophys. 42, 268, (1953)).
2.) Corticotensin: This peptide was reported to exist in porcine renal cortex (Fasciolo, J. R., Risler N. R., Totel G: Corticotensins: Pressor Peptides from the kidney. In: Genest J. and Koiw E. (editors) Hypertension 72. Berlin. Springer, p. 172ff.), and it also increases the blood pressure. It has not yet been isolated.
3.) Repeatedly, it was reported (Abelous J. E., Bardier E: in “De l´action de l´extrait alcoolique de l´urine humaine normale sur la pression artérielle” CR. Soc. Biol. 64, 596, 868 (1908), Bain W. in “The pressor basis of urine” J. Exp. Physiol. 8, 229 (1914), Bohn H., Hahn F. Z. in “Untersuchungen zum Mechanismus des blassen Hochdrucks, blutdrucksteigernde Stoffe im Harn, insbesondere beim blassen und roten Hochdruck” Zeit. Klin. Med. 123, 558 (1933)) that urine contained a substance having a pressor effect. In the meantime, this substance was described as a protein of 25,000 D molecular weight which has a hypertrophic effect on the renal cortex when administered chronically. The blood pressure increases, which also holds for plasma aldosterone and the Na⁺/K⁺ratio in the urine (Sen S., Bravo E. L., Bumpus F. M. in “Isolation of a hypertension producing compound from human urine” Circ. Res. 50, (suppl 1) 5 (1977)).
4.) Renopressin: It was reported that this protein was also contained in porcine renal cortex (Skeggs L. T., Kahn J. R., Levine M., Dorere F. E., Lentz K. E. in “Chronic one kidney hypertension in rabbits. III. Renopressin, a new hypertensive substance” Circ. Res. 40, 143 (1977)). These are experiments in which it could be shown that a formulation of renin-free porcine renal cortex slowly increases the blood pressure, and that this response could not be antagonized by angiotensin II blockers (Skeggs et al., supra).
5.) Nephrotensin: A hypertensive activity can also be recovered from the venous effluate of ischemic kidneys (Grollmann A. and Krishnamurty V. S. R. in “Differentiation of nephrotensin from angiotensin I and II” Proc. Soc. Exp. Biol. Med. 143, 85 (1973)). It has been asserted that this protein was angiotensin I bound to alpha-2-globulin (Schwikert J. R., Carey R. M., Liddle G. W., Islamnd D. P. in “Evidence that the renal pressor substance of Grollmann is related to angiotensin I” Circ. Res. 30, 31, 131 (1972)), but the activity is retained upon administration of an antibody against angiotensin I, and it is distinguished from the activities of angiotensin I and angiotensin II in rats (Grollmann, supra).

It has been an object of the present invention to provide further vasoactive substances. Surprisingly, by a special extraction method with subsequent separation, an as yet unknown hypotensive as well as a hypertensive activity could be recovered from mammal renal medulla, which is not identical with known substances having an effect on the blood pressure, such as prostaglandins E2, A2, prostacyclin or some other prostaglandin. Identity with the known medullipin could also be ruled out.

### DESCRIPTION OF THE FIGURES

FIG. 1 describes the hypotensive activity of the extract on spontaneously hypertensive (SH) rats at a dosage of 10 mg/kg of body weight. The arterial blood pressure (mm Hg) is plotted against time in minutes.

FIG. 2 describes the hypotensive activity of the fraction on normotensive rats at a dosage of 10 mg/kg of body weight. The arterial blood pressure (mm Hg) is plotted against time in minutes.

FIG. 3 describes the dose-dependence of the drop in blood pressure two minutes after the administration. The reduction of systolic and diastolic blood pressures (in mm Hg) is plotted for different dosages (5, 10 and 20 mg/kg of body weight).

FIG. 4 describes the dose-dependence of the drop in blood pressure one hour after the administration. The reduction of systolic and diastolic blood pressures (in mm Hg) is plotted for different dosages (5, 10 and 20 mg/kg of body weight).

FIG. 5 describes the hypertensive activity of the fraction on SH rats at a dosage of 10 mg/kg of body weight. The arterial blood pressure (mm Hg) is plotted against time in minutes.

FIG. 6 describes the effect of the solvent on SH rats. The arterial blood pressure (mm Hg) is plotted against time in minutes.

FIG. 7 describes the effect of an ineffective comparative fraction on SH rats. The arterial blood pressure (mm Hg) is plotted against time in minutes.

FIGS. 8a to 8d show the mass spectra of the prostaglandins employed as comparative compounds.

FIG. 9 shows the elution scheme, the relative weights of the individual fractions and the thin-layer chromatographic maps in the recovery of the hypotensive extract. TLC is an abbreviation of “thin layer chromatography”. Below, the numbers of the fractions are shown. Below, the relative weights of the individual fractions are stated as a bar diagram. Below, the amounts of solvent used are stated for each individual fraction.

### DESCRIPTION OF THE INVENTION

Hypotensive extract

Thus, in one aspect, the invention relates to a method for isolating a hypotensive activity from the renal medulla of mammals by
(i) drying the mammal renal medulla;
(ii) extracting the dried renal medulla with an organic solvent having a dielectric constant of between 2 and 45;
(iii) subjecting the extract obtained to chromatography over a hydrophilic sorbent using an eluent gradient of from non-polar to polar; and
(iv) recovering the hypotensive activity as an extract after separating off the eluent.

In connection with the invention, said “hypotensive activity, extract, fraction or substance” is to be understood as having the same meaning as “active substance”.

According to the invention, the mammal renal medulla can be obtained from pigs, rabbits, rats, sheep, dogs, bovines or primates.

The drying essentially serves for the technological improvement of the later extraction process and is characterized by allowing the mild removal of the water contained in the tissue. This is preferably effected by freeze-drying.

The organic solvent employed for extracting the renal medulla preferably has a dielectric constant of between 45 and 2. This corresponds to polarity indices of between 1.5 and 0 (according to M.K. Gosh in “Methods on Drug Analysis”, Springer Verlag, Heidelberg, 1992). In addition to supercritical carbon dioxide, there is preferred a C₁-C₃ chlorohydrocarbon selected from the group consisting of chloroform, methylene dichloride, 1,1-dichloroethane, 1,2-dichloroethylene and 1,1,2-trichloroethane. Chloroform is particularly preferred.

The sorbent used in column chromatography is preferably a normal phase selected from the group consisting of regular or irregular silica gels. Its grain size may be from 3 to 500 μm, preferably from 30 to 200 μm, more preferably from 45 to 100 μm. Its pore size is between 4 and 12 nm (40 and 120 Å), preferably between 6 and 12 nm (60-120 Å). However, it is also possible to use other hydrophilic (=polar) sorbents of non-derivatized or derivatized nature which are suitable for normal phase chromatography.

The eluent gradient is preferably produced by the eluent sequence: C₅-C₁₀ alkane, C₅-C₁₀, alkane/C₂-C₈ ether/ethanol, C₂-C₈ ether/ethanol, C₂-C₈ ether/ethanol/methanol, ethanol/methanol, and methanol, respectively in increasingly polar mixing ratios and mixtures. The organic solvents may also be replaced by appropriate solvents having a similar solvent strength and elution performance.

The mixing ratios (% v/% v) are preferably as follows: C₅-C₁₀alkane/C₂-C₈ ether/ethanol from 95/0/2 incrementally to 0/95/5, C₂-C8 ether/ethanol from 95/5 incrementally to 20/80, C₂-C₈ ether/ethanol/methanol from 20/80/0 incrementally to 0/50/50, ethanol/methanol from 50/50 incrementally to 0/100.

The C₅-C₁₀alkane is an aliphatic or alicyclic hydrocarbon and is preferably selected from the group consisting of n-pentane, n-hexane, cyclohexane, n-heptane, n- or iso-octane, n- or iso-nonane, n- or iso-decane. Particularly preferred is n-hexane.

The C₂-C₈ ether is aliphatic or cycloaliphatic and is preferably selected from the group consisting of diethyl ether, diisopropyl ether, ethyl isopropyl ether, tert-butyl methyl ether, tetrahydrofuran and dioxan. Particularly preferred is diethyl ether.

The alcohols to be preferably employed are selected from the group of C₁-C₄ n- or C₁-C₄ iso-alcohols, more preferably ethanol, methanol or mixtures thereof. Substitution of the pair ethanol/methanol is possible by an analogous combination of a rather lipophilic and a rather hydrophilic alcohol. The hypotensive activity is found in the polar fractions of gradient chromatography, i.e., in the fractions obtained by using the mentioned alcohols as eluents.

The column chromatography described herein can also be performed in an analogous way by reversing the polarity conditions of both the sorbent and the gradient using reversed phases as the sorbent. Particularly preferred are RP-18 and RP-8 silica gels having a grain size of from 3 to 500 μm, preferably from 30 to 200 μm, more preferably from 45 to 100 μm. The pore size is between 4 and 12 nm (40 and 120 Å), preferably between 6 and 12 nm (60-120 Å). However, it is also possible to use other hydrophilic (=polar) sorbents. Quite generally, column chromatography is a chromatography in open or closed columns which is driven by gravity of forced by reduced or increased pressure.

The invention further relates to a hypotensive extract obtainable by the method described above, and to a pharmaceutical composition comprising such hypotensive extract, or formulations which are incomplete in terms of the invention, especially formulations obtainable by omitting the drying step or omitting chromatography.

The hypotensive extract is preferably incorporated Into an aqueous phase. In addition to the hypotensively active substance, the ready-to-administer pharmaceutical formulation may further contain wetting agents, auxiliary solvents, solubilizers and stabilizers. The concentrations are dependent on the dose to be administered, which is stated in the following, and the volume or weight of the dosage form. The administration is effected on a systemic or peroral route.

The hypotensive extract described above and the pharmaceutical composition described above can be used for lowering the blood pressure in mammals, especially humans. Accordingly, the hypotensive extract and the pharmaceutical composition comprising said extract can be used for preparing a medicament for lowering the blood pressure in mammals.

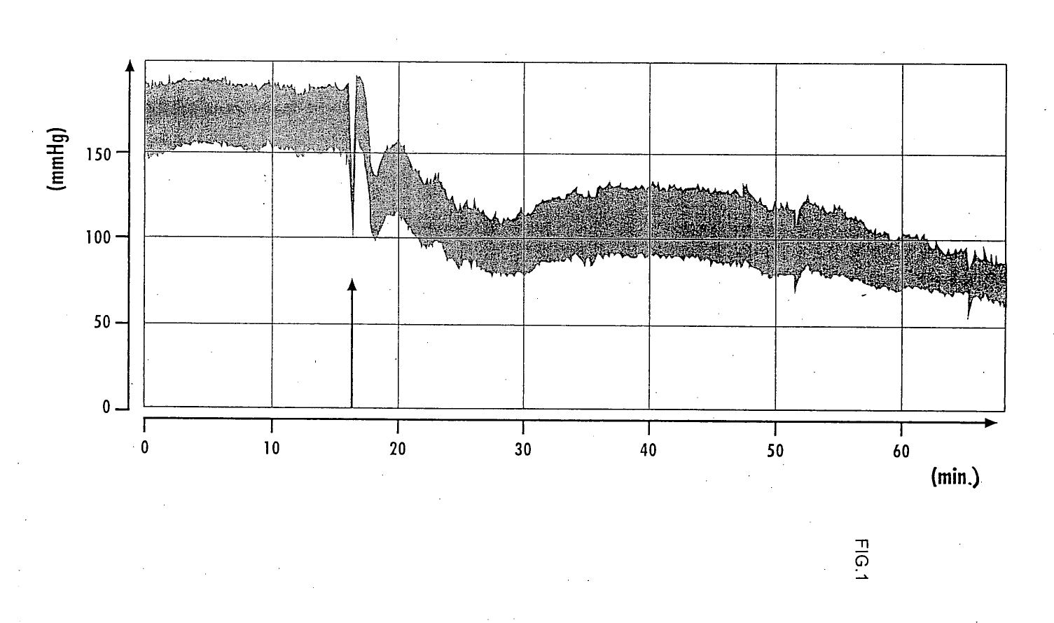

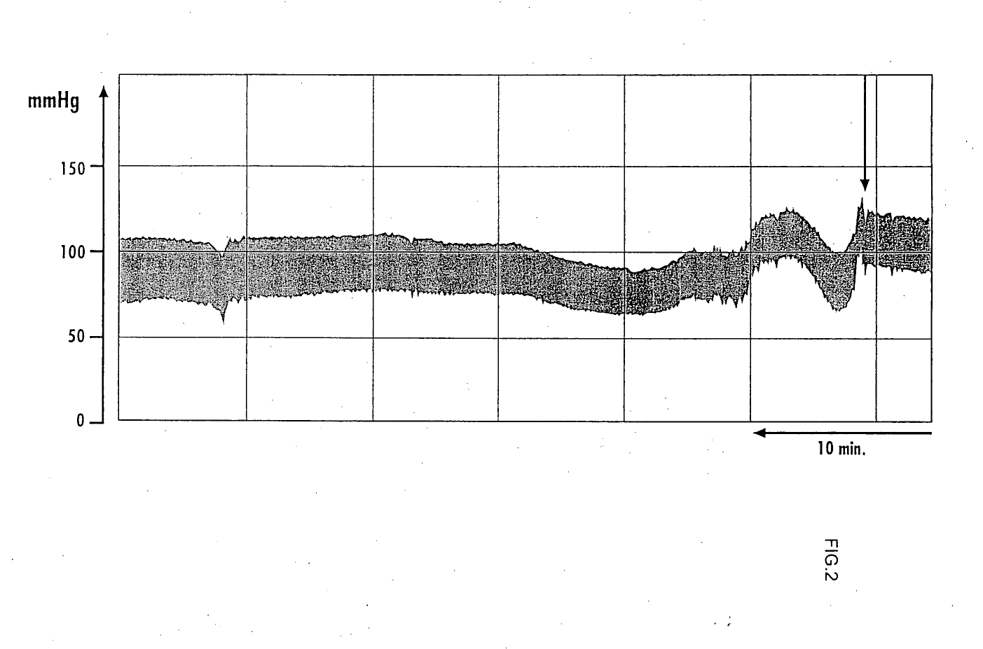

The dose to be administered is from 2 to 50 mg/kg of body weight, preferably 10 mg/kg of body weight, but may also have to be higher or lower. The effect of a dose of 10 mg/kg of body weight is shown in FIGS. 1 and 2.

Ten mg/kg of body weight was administered to a spontaneously hypertensive Wistar rat. The time of administration is indicated by the arrow. The blood pressure decreases, reaches a minimum after some minutes, slightly increases again, and again decreases. After one administration, the blood pressure may remain low for several hours or days.. This effect is observed in an analogous manner in a normotensive animal (FIG. 1 shows the drop in blood pressure in an SH rat).

Ten mg/kg of body weight was administered to a normotensive rat. The time of administration is indicated by the arrow. The blood pressure decreases, reaches a minimum after some minutes, slightly increases again, and again decreases. A second minimum is reached. The time to reach this minimum is more variable than the time to reach the first minimum. Thus, the time of the drop in blood pressure to the following rise is also variable. However, the similarity in principle of the blood pressure curve morphology in both Figures should be pointed out. The course of the drop in blood pressure is typical. After one administration, the blood pressure may be kept low for several hours or days. The chart in FIG. 2 is to be read from right to left. On the Y axis, the blood pressure is plotted against time on the X axis.

In a group of seven animals, the drop in blood pressure after this dose of the fraction was: 37.57±22.14/28.57±16.99 mm Hg after 2 minutes, and 57.42±29.07/45.57±22.41 mm Hg after one hour. This difference was statistically significant with p=0.0078.

In a group of seven animals, the drop in blood pressure after this dose of the extract before chromatography was: 20.71±5.37/17.71±6.72 mm Hg after two minutes, and 29.57±16.61/24.85±14.69 mm Hg after one hour. This difference was statistically significant with p=0.0078.

In a group of seven animals, the drop in blood pressure after the corresponding dose (1.5 ml) of the formulation without the mixture (ethanol/water) was: 0.71±2.92/−0.14±3.23 mm Hg after two minutes, and −1.14±4.67/−1.71±4.99 mm Hg after one hour. The blood pressure remained constant.

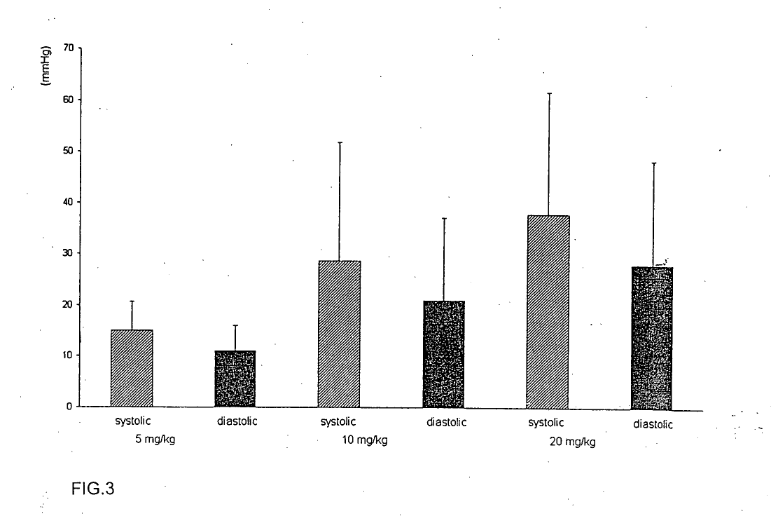

FIG. 3 shows the dose-dependence of the drop in blood pressure two minutes after the administration of the fraction. On the y axis, the drop in blood pressure is plotted against the dosages 5(n=5), 10(n=7) and 20(n=6) mg/kg of body weight on the x axis, separately for diastolic and systolic blood pressures. The drop in blood pressure is clearly dose-dependent.

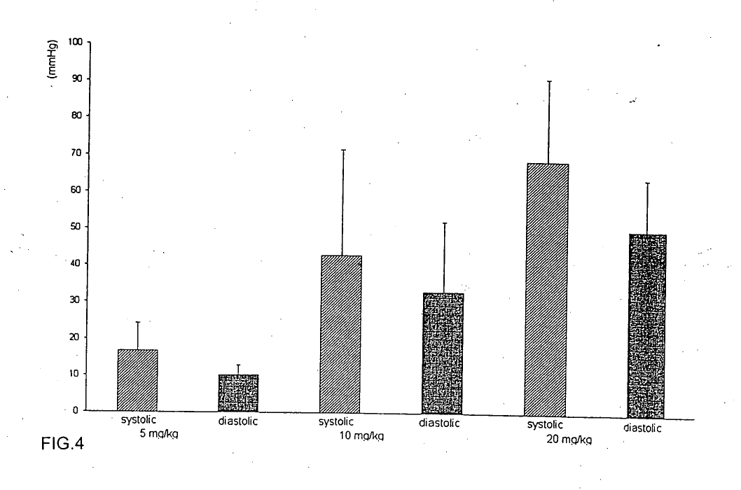

FIG. 4 shows the dose-dependence of the drop in blood pressure one hour after the administration of the fraction. On the y axis, the drop in blood pressure is plotted against the dosages 5(n=5), 10(n=7) and 20(n=6) mg/kg of body weight on the x axis, separately for diastolic and systolic blood pressures. The drop in blood pressure is clearly dose-dependent.

On the whole organism, the extract does not cause tachycardia during the hypotensive activity. Bradycardia has been observed.

The extract according to the invention is an endogenous substance of a mammal. No toxic effects have been observed.

As further galenic forms, there may be used all forms of injectables, oral dosage forms including those having a modified release of active ingredients, and dosage forms based on nanoparticles.

The turbidity, determined by photon correlation spectroscopy, of the injectable colloid of the extract which had been formulated as described below was caused by droplets having a size of 143±47 nm ( ). The colloid which had been formulated from fractions 47 to 73 had an average droplet size of 188±12 nm ( ). Thus, the particles had a droplet size which was far below the tolerance limit of lung penetration and were thus galenically unobjectionable for the experiment.

According to the present invention, it is shown that a renal medulla chloroform extract chromatographically processed as described above derived from a mammal, especially pig, is free from prostaglandins with a detection limit of better than 3 ppm, but simultaneously has antihypertensive properties. Converted for the animals, one animal cannot have contained more than about 0.6·10⁻¹⁰ M of a prostaglandin. This amount is irrelevant in terms of blood pressure and does not account for the effect showed herein. Thus, doubts about whether all hypotensive activities observed could be activities caused by prostaglandins after all have been dispelled. According to the invention, this activity is manifested almost acutely, has a maximum effect at about 10 minutes after the injection and all in all continues for more than one hour. For the activity according to the invention, there is no typical course of action which could be attributable to a known substance.

Thus, the hypotensively active substance according to the invention can be employed for dose-dependent lowering of the blood pressure in mammals or for achieving effects related to this activity, such as, in particular, relief of the heart and prevention or treatment of other cardiovascular diseases, irrespective of whether the initial blood pressure is a hypertensive blood pressure or a normotensive blood pressure. Continuous infusion of the active substance according to the invention is not necessary.

None of the hypotensive mixtures or substances of the prior art can possibly account for the activity observed according to the invention.

lso incapable of accounting for the hypotensive activity are prostanoid substances which are contained in the renal medulla in extraordinarily large amounts (Daniels et al., supra). However, these prostaglandins isolated from the renal medulla, prostaglandins E2 and A2, belong to the most potent vasodilatory agents known. However, it could be excluded that they could account for the activity described here by showing that they are not contained in sufficient amounts in the extract according to the invention. To isolate prostaglandins from renal medulla (Lee J. B., Crowshaw K., Takman B. H., Attrep K. A. in “The identification of prostaglandins E2, F2αa and A2 from Rabbit Kidney Medulla” Biochem. J.105; 1967: 1251-1260), the following method of lipid extraction as set forth by Lee (Lee J. B. in “Prostaglandins, neutral lipids, renal interstitial cells and hypertension” Genest. J., Koiw E., Kuchel O. (editors) in “Hypertension: Physiopathology and treatment” McGraw-Hill, New York, 1977, p. 373-390) was employed to exclude neutral lipids and to obtain the acidic prostaglandins while separating them from the neutral lipids. At first, acidified aqueous homogenizate of renal medulla was extracted with methylene chloride. It was expected that both the acidic and the neutral lipids would be contained. Then, the methylene chloride was shaken against a phosphate buffer with the idea that only the acidic, but not the neutral, lipids would be transferred into the buffer. The phosphate buffer was acidified and again extracted with methylene chloride; this solution then contained the activity which later resulted in detecting the prostaglandins mentioned in the kidney (Lee J. B., Crowshaw et al., supra).

If the extract described here contained prostanoid substances, an activity attributable to these substances would have to be expected in fractions 47 to 73 by analogy to the work by Lee. However, according to the invention, no such activity could be detected. After it has been shown that the renal medulla contains a significant amount of particularly potent vasodilatory prostaglandins, but none of these substances was detected in a sufficient amount according to the invention and no activity relating to the blood pressure and attributable to these substances was found, it is excluded that the hypotensive activity according to the invention in the fraction described according to the invention may be due to the presence of prostanoid substances.
The hypotensive extract is not due to vasodilatory and/or hypotensive prostanoid substances. The presence of these substances has been excluded, as shown above.
The activity of the hypotensive extract according to the invention is not due to the presence of a mixture called APRL since APRL is not present in the extract according to the invention (Prewitt R. L., Leach B. E., Byers L. W., Brooks B., Lands W. E. M., Muirhead E. in “Antihypertensive polar renomedullary lipid, a semisynthetic vasodilator” Hypertension 1, 1979: 299-308). This is because APRL is a semisynthetic mixture of lipids.
The activity of the hypotensive extract according to the invention is not due to the presence of medullipin, because:
medullipin was described to be AN(neutral)RL (Muirhead, in “textbook”, supra), but the activity according to the invention is to be characterized as polar to highly polar.
medullipin is eluted from a silica gel column with chloroform and can be found in the initial fractions (Muirhead E. E.., Byers L. W., Desiderio E. M., Pitcock J. A., Brooks B., Brown P. S., Brosius W. L. in “Derivation of antihypertensive neutral renomedullary lipid from renal venous effluent” J. Lab. Clin. Med. 99; 1982: 64-74).
medullipin is extracted by the method of Bligh and Dyer (Bligh E. G. and Dyer W. J. in “A Rapid Method of total lipid extraction and purification”, Canadian Journal of Biochemistry and Physiology: 37; (8) 1959), while the hypotensive activity according to the invention is extracted with chloroform.
An extract containing medullipin either does not display any activity on vessels, or else it displays a vasodilatory activity (Muirhead E. E., in “textbook”, supra, and Muirhead E. E. in “Depressor Functions of the Kidney” Seminars in Nephrology, Vol. 3, No. 1 (March), 14-29 (1983)). However, the vasodilatory activity to which the hypotensive effect of medullipin is attributed has never been shown. The hypotensive extract according to the invention is characterized, however, by a strong dilatory activity on the aorta of rats and coronary arteries of pigs.
The hypotensive extract according to the invention is among the agents which have the most potent known hypotensive activity in mammals. To lower the blood pressure almost immediately, strongly and with a sustained effect is a unique property by which the hypotensive extract according to the invention is also distinguished from all other known active substances or extracts.
The hypotensive extract according to the invention has a directly dose-dependent hypotensive effect, distinguishing it from medullipin or other renal medulla extracts for which such a property is not known.
The hypotensive extract according to the invention lowers the blood pressure not only in hypertensive individuals, but also to a similar extent in normotensive individuals, only depending on dosage. Such a property is otherwise found only in sodium nitroprusside; however, the latter must be infused continuously , i.e., the extract according to the invention has a long-lasting effect by which it is distinguished from the only comparable active substance, i.e., sodium nitroprusside, and which means a critical advantage over the latter. This is also a property not attributed to medullipin.
The blood pressure curve morphology found in connection with the hypotensive extract, i.e., the time course of the drop in blood pressure, is unique. It is distinguished from that of all known active substances or extracts:
From that of the prostaglandins because these cause an immediate (in the first seconds after administration) drop in blood pressure whose minimum is reached very quickly, after seconds to several seconds. As shown in the Figures, the activity according to the invention reaches a maximum not before some minutes later (=the first minimum of blood pressure), then the blood pressure increases again and drops to reach a second minimum. Prostanoid substances or any other known substances do not have such an effect. Prostaglandins lose their activity after a few minutes (Fitzpatrick T. H., Alter I., Corey E., Ramwell P., Rose J. C., Kot P. in Circulation Research 42; 1978: 192-194), which is not the case with the hypotensive activity according to the invention. In addition, the dose administered according to the invention without having detected it by GC-MS because it was below the detection limit is about three powers of ten lower than that required to produce an effect on blood pressure with prostaglandins (Fitzpatrick T. H., supra).
From that of medullipin since a medullipin extract is not immediately effective (with a time delay of some minutes) and a medullipin extract reaches only one minimum (Muirhead E. E., in “textbook”, supra, and Muirhead E. E. in “Depressor Functions of the Kidney”, Seminars in Nephrology, Vol. 3, No. 1 (March), 14-29 (1983)).
The hypotensive extract according to the invention did not induce tachycardia. This property is not found in any known endogenous or natural substance or any such mixture which is hypotensive. In the following, the hypotensive extract shall be referred to as “angiolysin”.

Hypertensive extract

In another aspect, the invention relates to a method for isolating a hypertensive activity from the renal medulla of mammals by
(i) drying the mammal renal medulla;
(ii) extracting the dried renal medulla with an organic solvent having a dielectric constant of between 2 and 45;
(iii) subjecting the extract obtained to chromatography over a hydrophilic sorbent using an eluent gradient of from non-polar to polar;
(iv) obtaining a residue after separating off the eluent; and
(v) recovering the hypertensive activity by subjecting the residue to gel chromatography with an organic solvent.

In connection with the invention, said “hypertensive activity, extract or substance” is to be understood as having the same meaning as “active substance”.

For the sources of renal medulla, the organic solvents used for its extraction, the performance of gradient chromatography and the elution of the residue of the subsequent gel chromatography, reference is made to the above statements made in connection with the hypotensive extract. The residue to be subjected to gel chromatography is obtained from the above described polar fractions (eluate fractions) of gradient chromatography.

For the gel chromatography, swellable polysaccharides, especially of the agarose and sepharose type, can be used as supports. Preferred is lipophilized and/or cross-linked sepharose of the Sephadex® type, such as Sephadex® LH-20. As the eluent, an organic solvent, preferably having a dielectric constant of between 2 and 45, is employed either in a pure form or as a mixture. As the eluent, there may be used solvents selected from the group consisting of C₁-C₄ alcohols, C₂-C₈ ethers, C₅-C₁₀ aliphatic alkanes, C₆-C₁₂ aromatic hydrocarbons, C₃-C₉ ketones and their mixtures. Preferably suitable are C₁-C₄ n- or C₁-C₄ iso-alcohols, especially ethanol or methanol, and C₁-C₃ chlorohydrocarbons. Particularly preferred are mixtures of C₁-C₄ n- or C₁-C₄ iso-alcohols, especially ethanol or methanol, with a C₁-C₃ chlorohydrocarbon in a mixing ratio of 0-100/0-100, preferably 30-70/30-70. The C₁-C₃ chlorohydrocarbon is selected from the above described group. Particularly preferred is a mixture of methanol and dichloromethane at a ratio of 1:1.

The invention further relates to a hypertensive extract obtainable by the method described above, and to a pharmaceutical composition comprising such hypertensive extract.

The hypertensive extract is preferably incorporated into an aqueous phase. In addition to the hypertensively active substance, the ready-to-administer pharmaceutical formulation may further contain wetting agents, auxiliary solvents, solubilizers and stabilizers. The concentrations are dependent on the dose to be administered, which is stated in the following, and the volume or weight of the dosage form. The administration is effected on a systemic or peroral route.

The hypertensive extract described above and the pharmaceutical composition described above can be used for raising the blood pressure in mammals, especially humans, and accordingly, the hypertensive extract or the pharmaceutical composition are used for preparing a medicament for raising the blood pressure in mammals. The dose to be administered is from 2 to 100 mg/kg of body weight, preferably 10 mg/kg of body weight, but may also be higher or lower, depending on the patient's response.

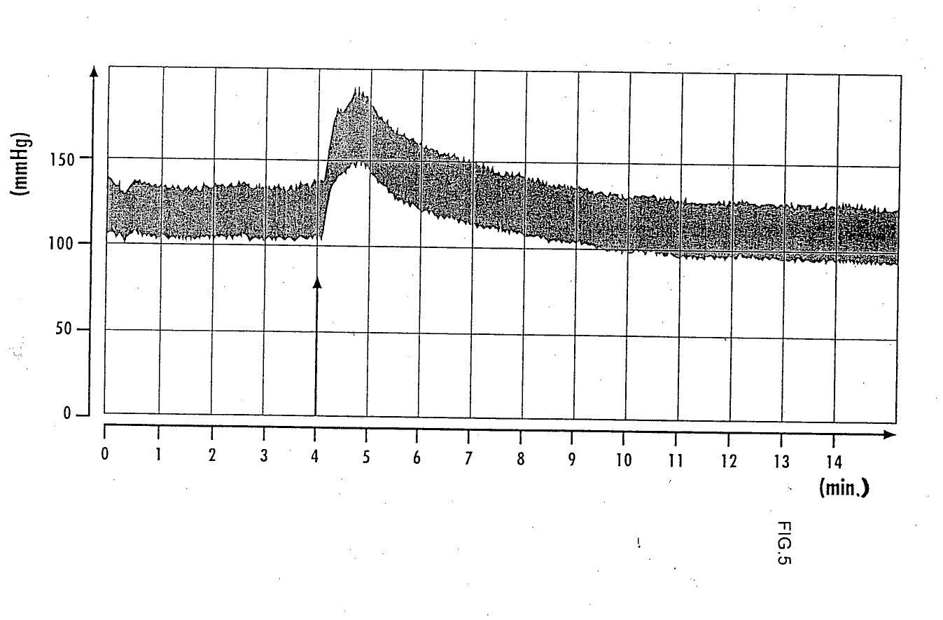

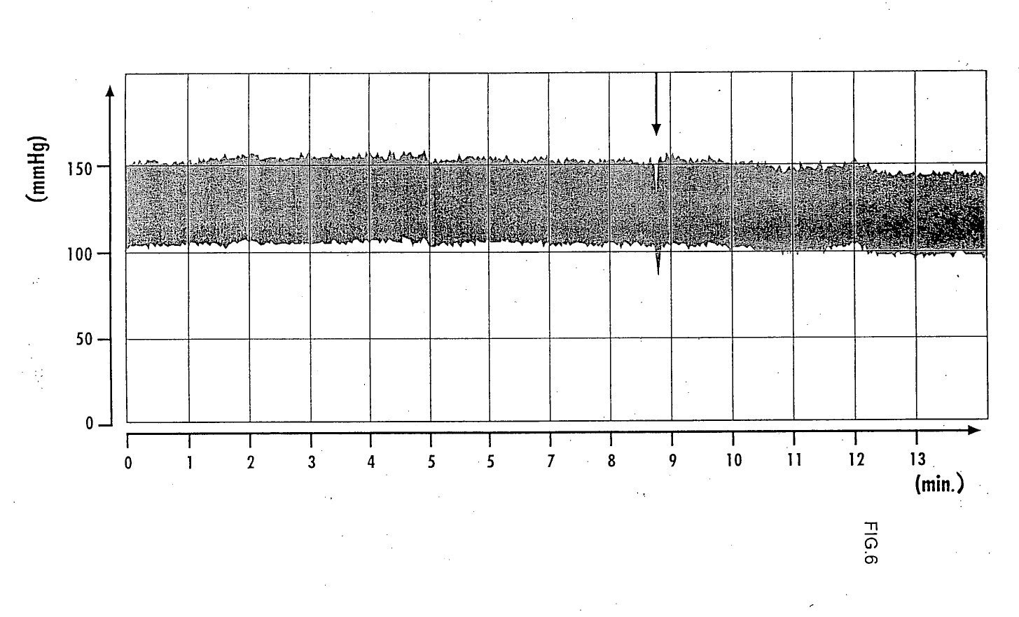

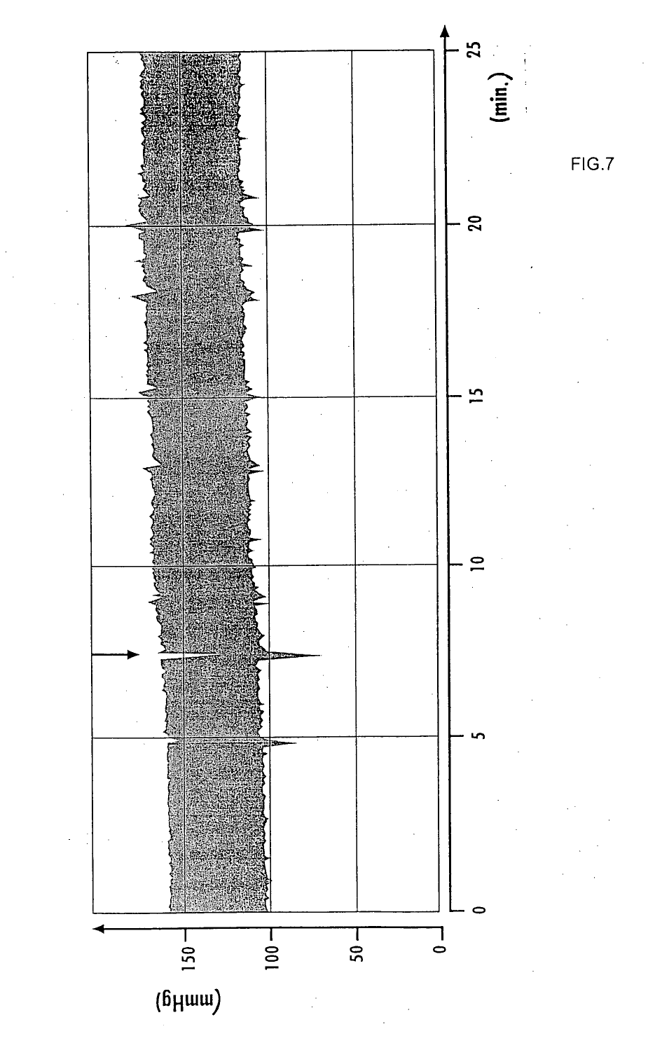

The effect of a dose of 10 mg/kg of body weight is shown in FIG. 5. An immediate rise in blood pressure occurs which remains for some minutes. The effect of the corresponding solvent is shown in FIG. 6. The effect of a comparative fraction which is ineffective is shown in FIG. 7.

Thus, the effects shown, both hypertensive and hypotensive, are not due to a volume effect of the solvent, as shown here. The effects shown, both hypotensive and hypertensive, are not due to properties of the formulation, as shown here.

The description of the groups which were formed is as follows:
1.) Fractions 1 to 276: This fraction shows a pronounced hypertensive activity which reaches its maximum at 55±27 seconds after completion of the injection and lasts for 201±59 seconds. An example of this short-term rise in blood pressure is shown in FIG. 1. As can be seen therefrom, the blood pressure starts to rise continuously almost immediately after the injection, reaches its maximum and decreases again somewhat more slowly to reach the original level. The rise in blood pressure was 30.1±7.1 mm Hg systolic and 34.7±6 mm Hg diastolic (p=0.0003).
2.) Fractions 1 to 48 (comparative fraction): After injection of this fraction, a significant change in blood pressure could not be observed. An example is shown in FIG. 2, which also proves that no visible change occurs. At the time which corresponds to the peak of blood pressure observed after the injection of fractions 1 to 276, i.e., 55 seconds after injection, only a small and statistically insignificant decrease in systolic pressure by 1.1±3 mm Hg was observed in the comparative fraction, accompanied by a small and statistically insignificant decrease in diastolic pressure by 0.3±5.1 mm Hg.
3.) Solvent control: After the injection of a mixture of ethanol and water as used for formulating the injectables of the samples according to items 1.) and 2.), a significant effect on blood pressure could not be observed either. An example is shown in FIG. 2. At the time which corresponds to the peak of blood pressure observed after the injection of fractions 1 to 276, i.e., 55 seconds after injection, only a small and statistically insignificant decrease in systolic pressure by 0.1±3 mm Hg was observed in the solvent control, accompanied by a small and statistically insignificant decrease in diastolic pressure by 0.9±2.2 mm Hg.

Further galenic forms include all forms of injectables, oral dosage forms including those having a modified release of active ingredients, and dosage forms based on nanoparticles. The hypertensive activity according to the invention represents a lipid contained in the renal medulla of the above mentioned mammals, especially pigs, which is capable of raising the blood pressure for some minutes. To date, apart from the gas-chromatographic detection of thromboxan A2 (Goswami S., Mai I., Bruckner G., Kuinsella J. E. in “Extraction and Purification of prostaglandins and thromboxane from biological samples for gas chromatographic analysis” Prostaglandins 22; 1981, 693-702), such a hypertensive lipid has not been reported to occur in the kidney.

In all other cases, proteins are involved which in part even have a rather high molecular weight. According to the invention, however, the renal medulla was extracted with pure chloroform; this does not correspond to the method of lipid extraction and purification of Bligh and Dyer (supra), in which from a monophasic mixture of tissue fluid, methanol and chloroform in which the tissue is homogenized, a biphasic mixture is prepared by adding water and chloroform, the chloroform layer containing the lipids and the methanol/water layer containing all other substances, which was employed by Muirhead.

A substance which could possibly be responsible for the hypertensive activity described according to the invention is thromboxan A2 as an endogenous peroxide derived from arachidonic acid. The activity isolated according to the invention is best comparable with the hypertensive property of this peroxide in terms of intensity and duration, despite some deviations, especially the faster rise in blood pressure after the administration of thromboxan A2 (Svensson, J. in “Biosynthesis and biological properties of prostaglandin endoperoxides and thromboxane A2”, Acta Biol. Med. Germ. 37, 1978: 731-740). However, due to its low stability with a half life of only about 30 seconds, it can be ruled out as a candidate with certainty. Under the conditions according to the invention for isolation and processing, thromboxan A2 would have been destroyed with certainty (Svensson et al., supra).
The hypertensive extract according to the invention is not derived from the catecholamines and increases the blood pressure immediately and briefly, but strongly. This renders it excellently controllable during application.
The hypertensive extract according to the invention causes an increase of blood pressure also for normal or low blood pressures.
The morphology of the blood pressure curve, i.e., the time course of the rise in blood pressure, is unique and has not yet been described. In the following, the hypertensive extract shall be referred to as “medullopressin”.

The following Examples describe the invention, but without limiting it.

### EXAMPLES

1. Hypotensive Activity (Angiolysin)

Materials and Methods

(i) Recovery of the renal medulla

The renal medulla was recovered from porcine kidneys. About 15 minutes after the animals had been slaughtered, immediately after meat inspection, the kidneys were separated from the lung-liver-kidney package on the conveying belt with a knife and immediately placed into crushed ice. About 45 minutes later, they were shock-frozen in liquid nitrogen and stored at −40° C. until dissection.

For dissection, the kidneys were warmed at room temperature until a medullar temperature of from −5 to −2° C. was reached. In this condition, it was possible to cut them lengthwise into about 2-3 mm thick slices without thawing using a conventional electric universal slicing machine.

From the kidney slices, as quickly as possible and very carefully, the renal medulla was separated from the renal pelvis and renal cortex using scalpels and immediately replaced into liquid nitrogen. Subsequently, it was again stored at −40° C. In this way, 23.42 kg of renal medulla was obtained from about 2500 kidneys (800 kg).

The renal medulla was subsequently powderized. Thus, in a frozen state, it was added to the nitrogen-cooled steel cup of a kitchen mixer in small portions of 80 to 100 g each. On top of the renal medulla, liquid nitrogen was also added to the steel cup. As soon as the latter had evaporated, the renal medulla was powderized. The same procedure was applied to the whole material portion by portion. The powderized renal medulla was again stored at −40° C.

Subsequently, the powder was freeze-dried in the conventional manner. The dry mass of the freeze-dried material was 3.62 kg.

(ii) Extraction

To this end, 1585.5 g of the lyophilizate was transferred into a 20 liter beaker, covered with chloroform and mixed with a mixing stick at the highest stage for about 10 minutes. Subsequently, the removal of suspended matter was effected by filtration with a conventional paper filter. The filtered-off material was now extracted in the same way nine more times with a total of about 75 liters of chloroform until the filtrate, which had a dark yellowish color in the first extraction steps, had become colorless. The solution obtained was then brought to dryness in vacuo at room temperature. The residue was 204.9 g of a highly viscous dark yellowish oil. For test purposes, a sample of 2 g weight was removed therefrom. The material was then charged with some chloroform onto 259.9 g of irregular silica gel supplied by ICN and having a grain size of from 32 to 64 pm and a pore size of 600 nm (60 Å) and again freed from the chloroform in a desiccator to obtain 464 g of a dry powder which was stored at −80° C. until further processed.

(iii) Gradient Chromatography

In order that the separation need not be divided into several steps, but because the column dimensions then required no longer allow a gravity-driven elution, a vacuum chromatographic column for elution was designed and constructed. In this column, elution can be performed using a vacuum in such a way that the ratio of sorbent to sample can be increased in favor of the amount of sorbent employed. The column employed had a height of 400 mm and an inner diameter of 16 mm and was filled with 2204 g of silica gel supplied by ICN having a grain size of 32 to 64 pm and a pore size of 600 nm (60 Å). Onto the extract, a paper filter and 200 g of sea sand were placed. Elution was effected in 73 steps over a gradient, beginning with hexane through hexane-diethyl ether, hexan-diethyl ether-ethanol, diethyl ether-ethanol, diethyl ether-ethanol-methanol, ethanol-methanol to pure methanol.

(iv) Elution of the Column

Fraction 73 consisted of pure chloroform since chloroform had been used for extracting. Elution was started with pure hexane. 73 fractions were obtained. The exact volumes of the 73 fractions are shown in the following Table:

TABLE

| | Hexane | Diethyl ether | Ethanol | Methanol | Chloroform |
|---|---|---|---|---|---|
| | (ml) | (ml) | (ml) | (ml) | (ml) |
| Fraction 1 | 1500 | 0 | 0 | 0 | 0 |
| Fraction 2 | 980 | 20 | 0 | 0 | 0 |
| Fraction 3 | 970 | 30 | 0 | 0 | 0 |
| Fraction 4 | 960 | 40 | 0 | 0 | 0 |
| Fraction 5 | 950 | 50 | 0 | 0 | 0 |
| Fraction 6 | 940 | 60 | 0 | 0 | 0 |
| Fraction 7 | 1395 | 105 | 0 | 0 | 0 |
| Fraction 8 | 1380 | 120 | 0 | 0 | 0 |
| Fraction 9 | 1365 | 135 | 0 | 0 | 0 |
| Fraction 10 | 1350 | 150 | 0 | 0 | 0 |
| Fraction 11 | 890 | 100 | 10 | 0 | 0 |
| Fraction 12 | 880 | 110 | 10 | 0 | 0 |
| Fraction 13 | 870 | 120 | 10 | 0 | 0 |
| Fraction 14 | 1290 | 195 | 15 | 0 | 0 |
| Fraction 15 | 1275 | 210 | 15 | 0 | 0 |
| Fraction 16 | 840 | 150 | 10 | 0 | 0 |
| Fraction 17 | 830 | 160 | 10 | 0 | 0 |
| Fraction 18 | 820 | 170 | 10 | 0 | 0 |
| Fraction 19 | 810 | 180 | 10 | 0 | 0 |
| Fraction 20 | 800 | 190 | 10 | 0 | 0 |
| Fraction 21 | 790 | 200 | 10 | 0 | 0 |
| Fraction 22 | 780 | 210 | 10 | 0 | 0 |
| Fraction 23 | 760 | 230 | 10 | 0 | 0 |
| Fraction 24 | 730 | 260 | 10 | 0 | 0 |
| Fraction 25 | 700 | 280 | 20 | 0 | 0 |
| Fraction 26 | 680 | 300 | 20 | 0 | 0 |
| Fraction 27 | 660 | 320 | 20 | 0 | 0 |
| Fraction 28 | 630 | 340 | 30 | 0 | 0 |
| Fraction 29 | 620 | 350 | 30 | 0 | 0 |
| Fraction 30 | 600 | 370 | 30 | 0 | 0 |
| Fraction 31 | 580 | 390 | 30 | 0 | 0 |
| Fraction 32 | 560 | 410 | 30 | 0 | 0 |
| Fraction 33 | 540 | 420 | 40 | 0 | 0 |
| Fraction 34 | 520 | 440 | 40 | 0 | 0 |
| Fraction 35 | 750 | 690 | 60 | 0 | 0 |
| Fraction 36 | 480 | 480 | 40 | 0 | 0 |
| Fraction 37 | 690 | 750 | 75 | 0 | 0 |
| Fraction 38 | 430 | 520 | 50 | 0 | 0 |
| Fraction 39 | 400 | 550 | 50 | 0 | 0 |
| Fraction 40 | 350 | 600 | 50 | 0 | 0 |
| Fraction 41 | 450 | 975 | 75 | 0 | 0 |
| Fraction 42 | 375 | 1050 | 75 | 0 | 0 |
| Fraction 43 | 200 | 750 | 50 | 0 | 0 |
| Fraction 44 | 175 | 750 | 75 | 0 | 0 |
| Fraction 45 | 150 | 750 | 100 | 0 | 0 |
| Fraction 46 | 75 | 825 | 100 | 0 | 0 |
| Fraction 47 | 0 | 900 | 100 | 0 | 0 |
| Fraction 48 | 0 | 750 | 250 | 0 | 0 |
| Fraction 49 | 0 | 1050 | 450 | 0 | 0 |
| Fraction 50 | 0 | 900 | 600 | 0 | 0 |
| Fraction 51 | 0 | 750 | 750 | 0 | 0 |
| Fraction 52 | 0 | 250 | 750 | 0 | 0 |
| Fraction 53 | 0 | 375 | 1050 | 75 | 0 |
| Fraction 54 | 0 | 250 | 600 | 150 | 0 |
| Fraction 55 | 0 | 250 | 500 | 250 | 0 |
| Fraction 56 | 0 | 150 | 500 | 350 | 0 |
| Fraction 57 | 0 | 50 | 500 | 450 | 0 |
| Fraction 58 | 0 | 50 | 500 | 500 | 0 |
| Fraction 59 | 0 | 50 | 500 | 500 | 0 |
| Fraction 60 | 0 | 125 | 525 | 750 | 0 |
| Fraction 61 | 0 | 50 | 350 | 600 | 0 |
| Fraction 62 | 0 | 0 | 350 | 650 | 0 |
| Fraction 63 | 0 | 0 | 250 | 750 | 0 |
| Fraction 64 | 0 | 0 | 200 | 800 | 0 |
| Fraction 65 | 0 | 0 | 150 | 850 | 0 |
| Fraction 66 | 0 | 0 | 100 | 900 | 0 |
| Fraction 67 | 0 | 0 | 0 | 1000 | 0 |
| Fraction 68 | 0 | 0 | 0 | 1000 | 0 |
| Fraction 69 | 0 | 0 | 0 | 1000 | 0 |
| Fraction 70 | 0 | 0 | 0 | 1000 | 0 |
| Fraction 71 | 0 | 0 | 0 | 1000 | 0 |
| Fraction 72 | 0 | 0 | 0 | 1000 | 0 |
| Fraction 73 | 0 | 0 | 0 | 0 | 1000 |

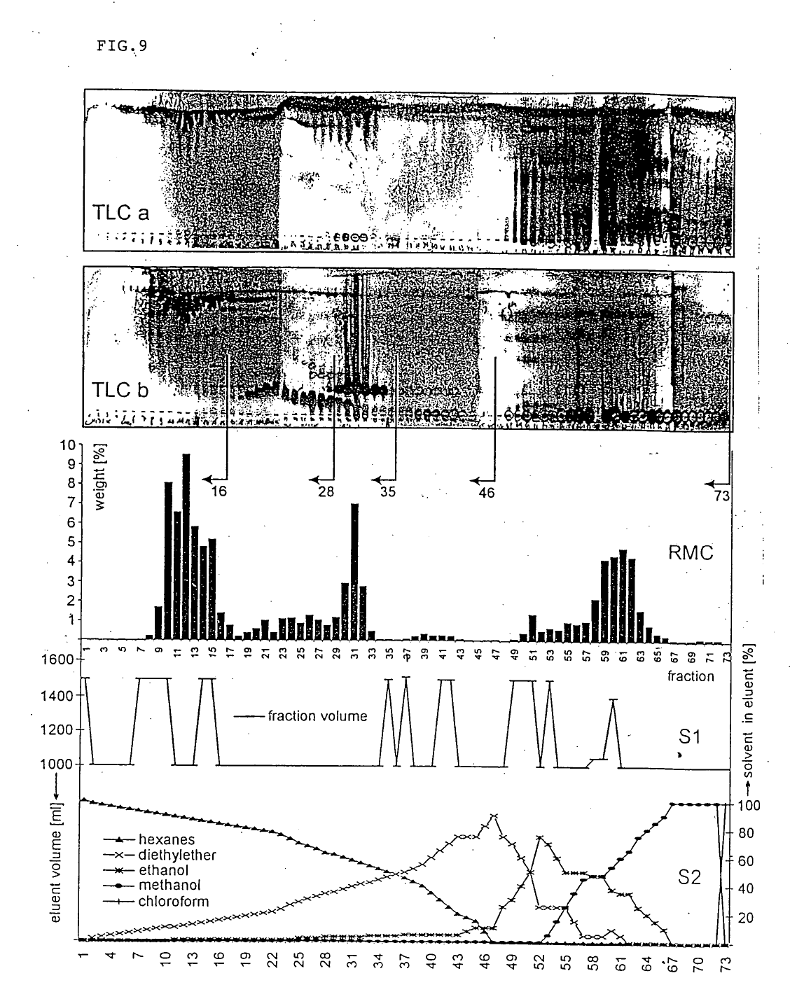

A portion of 50 μl of all fractions was applied to two thin-layer chromatographic plates each. The thin-layer chromatographic plates were prefabricated precoated silica plates having a size of 10*20 cm (Merck, Darmstadt, Germany). As the solvent, chloroform/methanol/water(70:29: 1) was used forone plate, and purechloroform for the other plate. The plates were developed with the anisaldehyde/sulfuric acid reagent according to DAB. The elution scheme, the relative weights of the individual fractions and the thin-layer chromatographic maps are shown in FIG. 9.

The 73 fractions were combined to pooled fractions according to thin-layer chromatographic aspects to form pooled fractions 1 to 16, 17 to 28, 29 to 35, 36 to 46, and 47 to 73. This division is set forth in FIG. 9. From each fraction, aliquots of 2% each (v/v) were withdrawn for performing the animal experiments and for gas chromatography/mass spectrometry.

(v) Gas Chromatography-Mass Spectrometry (GC-MS); Establishing of a Method for the Trace Detection of Prostaglandins A₂, E₂, F₂ₐ and I₂ (Prostacyclin)

The comparative prostaglandins were: Prostaglandin A₂ ([5Z,13E,15S]-15-hydroxy-9-oxoprosta-5,10,13-triene-1-oic acid), E₂ ([5Z,11a,13E,15S]-11,15-dihydroxy-9-oxoprosta-5,13-diene-1-oic acid) and F₂ₐ ([5Z,9a,13E,15S]-9,11,15-trihydroxyprosta-5,13-diene-1-oic acid) supplied by Sigma, St. Louis (Mo., U.S.A.); prostacyclin (syn. prostaglandin I_{2,} [5Z,9a,11a,13E,15S]-6,9-epoxy-11,15-dihydroxyprosta-5,13-diene-1-oic acid) supplied by ICN Biomedicals Inc., Eschwege (Germany).

For the derivatization of the pure substances and the samples as TMS ethers, a mixture of BSTFA and TMCS in pyridine was employed according to the manufacturer's protocol (Macherey & Nagel, Duren, Germany). Typically, 1 mg of comparative substance or the aliquots of the individual fractions (about 1-5 mg) concentrated to dryness was admixed with 90 ml of BSTFA, 30 ml of TMCS and 30 ml of pyridine and heated to 120° C. for 30 minutes in a tightly sealed microvial. In the case of the pure substances, from the thus obtained stock solutions (6.7 mg/ml), dilutions were prepared for GC at ratios of 1:20 (333 ng/ml) and 1:100 (67 ng/ml) using BSTFA reagent solution as a diluent. It was found that the decrease of the total ion chromatogram (TIC) in the chromatograms was directly proportional to the decrease of the concentrations of the substances in the solutions.

As the instrument, a GC-Q supplied by Finnigan MAT, Bremen (Germany), with a GC column of the type DB-5 was employed. The GC temperature program started at 100° C. with a holding time of one minute, increased to 280° C. with a heating rate of 8° C./minute and held this final temperature for 20 minutes. The injection volume was 1 ml with a split (1:20) for the comparative substances and without a split for the analytical samples. The MS conditions were as follows: Accelerating voltage of 70 keV, measurement in a positive EI mode within a range of m/z=50 to 1000 with 3 microscans, source temperature 200° C., temperature of the transfer line 220° C. Using prostaglandin E₂ as an example, the number of rinsing cycles of the injection device was established which were necessary for certainly no longer being able to detect the substances in such measurements which immediately follow the measurement of the comparative substances. Such detection could be just still furnished after the injection of undiluted silylation mixtures of prostaglandin E₂ after three regular rinsing cycles with dichloromethane. Thereupon, the number of rinsing cycles after each injection was set to five. In addition, all further measurements of comparative substances were performed with solutions diluted to at least 1:20, so that the possibility of cross-contamination was excluded.

At a GC total running time of 43.5 minutes and considering extraction of solvent (4 minutes), MS chromatograms having a time resolution of 0.99 seconds were obtained in this way. The average variation of the retention times for the comparative substances was 1.0 second, thus exactly corresponding to a mass scan. For the comparative substances, the following retention times were determined: prostaglandin A₂ 20.37 minutes, prostaglandin E₂ 21.51 minutes, prostaglandin F₂ₐ 21.32 minutes, prostaglandin I₂ (prostacyclin) 22.06 minutes. The following Table I summarizes the characteristic mass fragments recorded above m/z=200 which were recurred to for the selective search for prostaglandins by establishing individual mass traces of the GC chromatograms, respectively based on one of the characteristic masses. The efficiency of this approach can be seen from the following facts: On the one hand, there is a striking similarity of the fragment formation (→universal mass traces at, for example, m/z 227, 317, 388) for the prostaglandins A₂, E₂ and I_{2,} but on the other hand, there are also certain analogies of fragment formation for prostaglandin F₂ₐ. Accordingly, in addition to establishing the above mentioned universal mass traces, detection was made with an enhanced tolerance width [→“range”] at m/z=462/463 and 552/553, and including additional individual masses (→m/z 391, 537), the selective covering of typical fragments of prostaglandin F₂ₐ was effected.

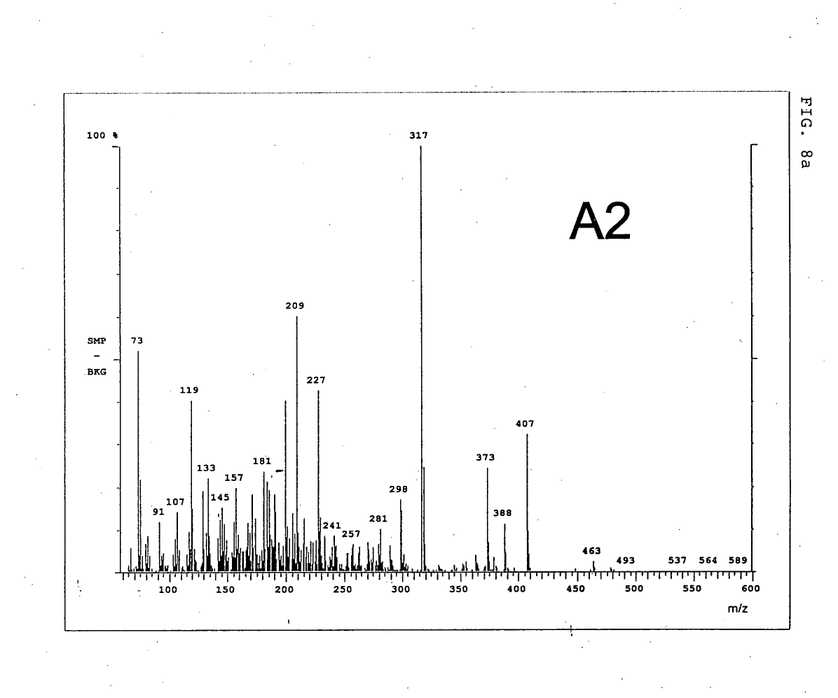

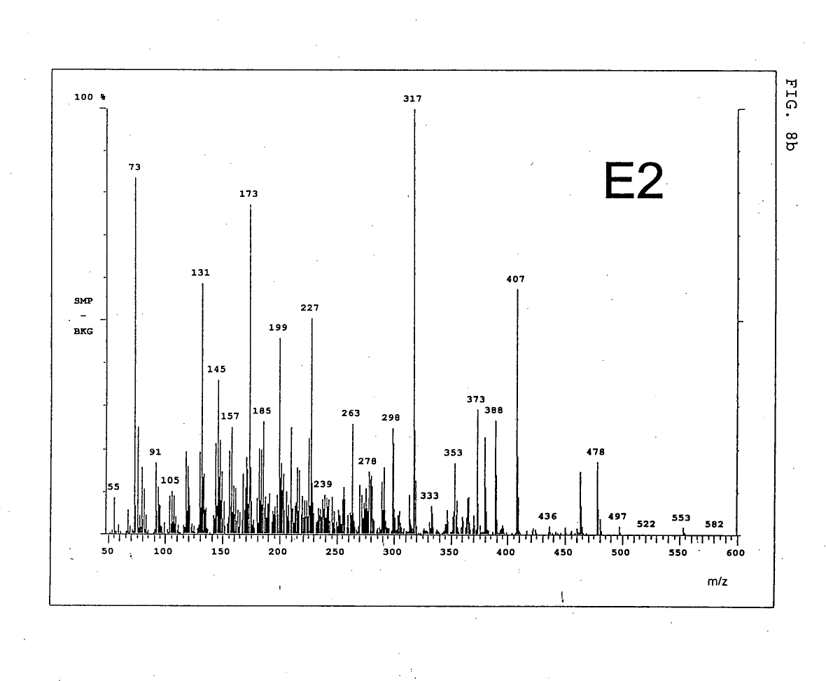

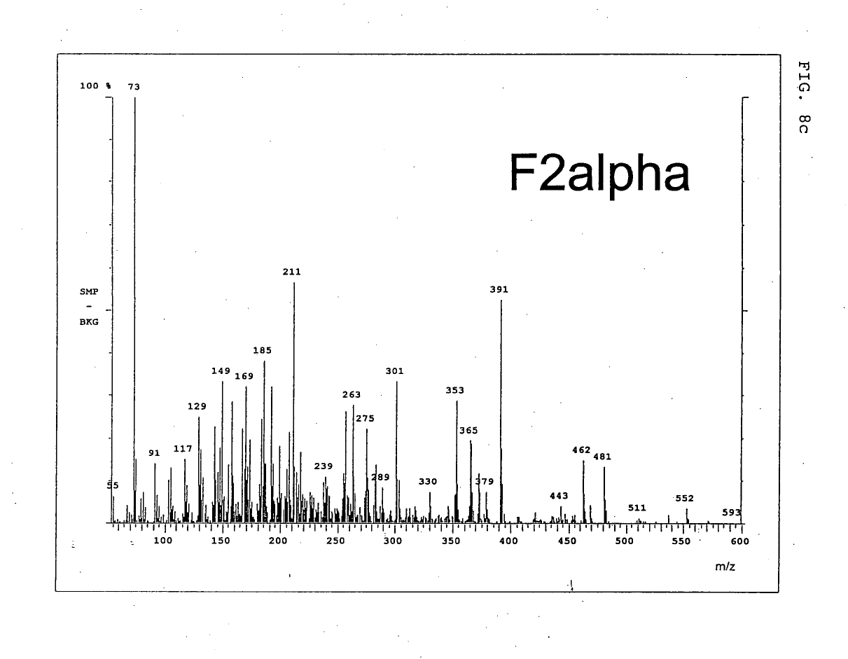

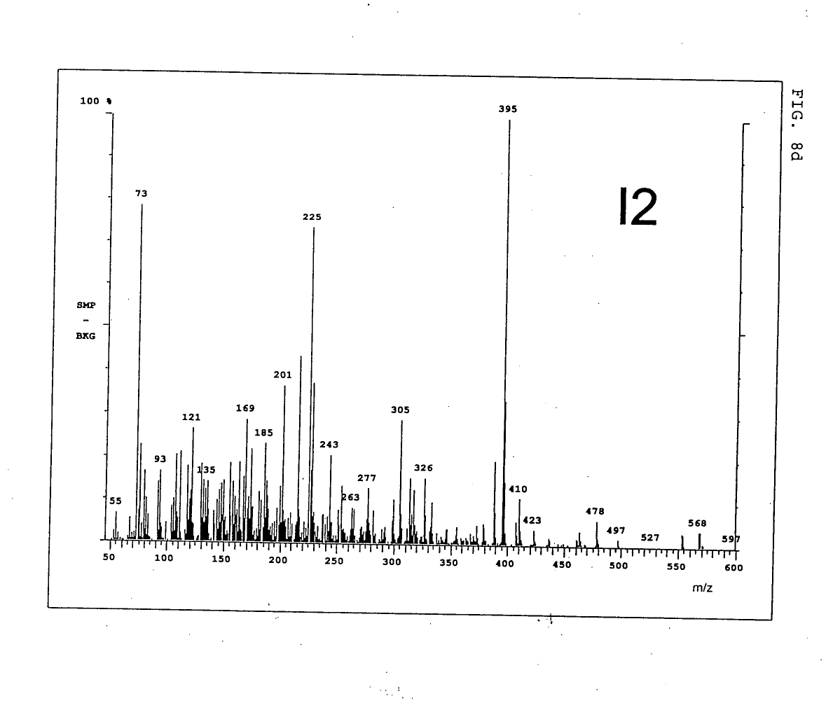

The related mass spectra are shown in FIGS. 8a ; to 8d.

TABLE 1
Characteristic mass fragments of the TMS ethers of the examined pure
prostaglandins A₂, E₂, F₂ₐ and I₂.

| | | | Typical fragmentary ions [m/z] |
|---|---|---|---|
| Prostaglandin | t_{R} [min] | M⁺ [m/z] | above 200 au |
| A₂ | 20.37 | 478 | 227, 317, 373, 388, 407, 463 |
| E₂ | 21.51 | 568 | 227, 317, 373, 388, 391, 407, 463, |
| | | | 478 |
| F₂ₐ | 21.32 | 642 | 211, 301, 353, 372, 391, 462, 481, |
| | | | 537, 552 |
| I₂ | 22.06 | 568 | 227, 317, 388, 395, 463, 478, 553 |

(vi) Detection of the Various Prostaglandins MS in the Test Fractions using Gas Chromatography/Mass Spectrometry (GC-MS)

The concentration of the diluted reference solutions for the prostaglandins was 333 ng/ml (1:20) and 67 ng/ml (1:100); the concentrations of the TMS derivatization mixtures prepared from the aliquots from the 73 individual fractions were about 6700-20,000 ng/ml each. Including the dynamic measuring range of the GC-MS measurements, the detection limit for the prostaglandins in the fractions can thus be calculated to be 2.2 ppm. The dynamics of mass-spectrometric identification after separation which underlie this calculation were better than 1:40; this value was calculated as the relation of the minimum necessary area, proportional to the ion current, of a peak in the mass chromatogram with which a mass spectrum could be achieved that was still recognized with certainty as being identical with the authentic comparative spectrum. Converted for the animals, one animal cannot have contained more than about 0.6·10⁻¹⁰ M of a prostaglandin in the experiment according to the invention. This amount is irrelevant in terms of blood pressure and does not account for the effect showed herein.

(vii) Formulation of the Samples for the Animal Experiments

To prepare the samples for the animal experiments, aqueous suspensions of the fat-like residues were prepared in commercially available aqua ad injectabilia with the aid of ethyl alcohol. Thus, the fat-like residue was first wetted with the ethanol and filled with the water at a ratio of 1:19 (v/v). This mixture was then homogenized using a commercially available ultrasonic microsuspension device once to three times for 15 to 30 seconds each at 5 to 30 Watt output power.

(viii) Establishing of the Average Particle Size and the Size Distribution of the Injected Suspensions using Photon Correlation Spectroscopy (PCS).

Determination of the particle size distribution in the aqueous suspensions prepared by microsuspension was effected by means of photon correlation spectroscopy (PCS) using an Autosizer supplied by Malvern Instruments (Malvern Autosizer 2c series 7032, Herrsching, Germany), equipped with a Multi-8 Correlator. The values for the size of the colloidal particles as determined by turbidimetry are respectively stated as mean values and standard deviations of five separate measurements. The turbidity, determined by photon correlation spectroscopy, of the injectable colloid of the extract prepared as described above was caused by droplets having a size of 143±47 nm. The colloid which had been prepared from fractions 47 to 73 had an average particle size of 188±12 nm. Thus, the particles had a droplet size which was far below the tolerance limit of lung penetration and were thus galenically unobjectionable for the experiment.

(ix) Animal Experiments

For the animal experiments, adult spontaneously hypertensive Wistar rats with an average weight of about 400 g were used. Under Ketanest/diazepam anesthesia, the individual doses (10 mg/kg of body weight) with a volume of 1500 pl were injected within 10 seconds through the vena jugularis into the vena cava superior. Measurement of the blood pressure was effected intraarterially in the arteria iliaca communis using a pressure transducer in connection with a Siemens Sirecust 404 monitor. For calibrating the pressure values, a mercury gauge according to Gauer was used. Recording of the blood pressure was performed analogously on a commercially available recorder as well as digitally. The monitoring period was one hour each. In a group of seven animals, the drop in blood pressure after a dose of 10 mg/kg of body weight of the extract prior to chromatography was: 20.71±5.37/17.71±6.72 mm Hg after two minutes, 29.57±16.61/24.85±14.69 mm Hg after one hour. This difference was statistically significant with p=0.0078.

In a group of seven animals, the drop in blood pressure after a dose of 10 mg/kg of body weight of fractions 47-73 was: 37.57±22.14/28.57±16.99 mm Hg after 2 minutes, and 57.42±29.07/45.57±22.41 mm Hg after one hour. This difference was statistically significant with p=0.0078.

Thus, both the extract and the pooled fractions 47-73 had the effect described above.

2. Hypertensive Activity (Medullopressin)

To obtain the hypertensive activity, the same procedure was used as described above under item 1., Materials and methods (i) to (iv), in connection with the recovery of the hypotensive activity.

(x) Gel Chromatography

The two fractions mentioned last in said section (cf. item iv) were further separated by gel chromatography on Sephadex LH-20 (Fluka, Buchs, Switzerland). For this purpose, for fractions 36 to 46, a column filled 143 cm high and having an inner diameter of 4.2 cm and a filling quantity of about 500 g was used; for fractions 47 to 73, it was a column filled 95 cm high and having an inner diameter of 6.3 cm and a filling quantity of about 700 g. Elution was respectively performed with a mixture of methanol and dichloromethane (50/50; v/v). The activity was found in fractions 1 to 276, obtained from fractions 47 to 73 of the silica gel column; the volume of the fractions was 12 ml each. The corresponding fractions, the also early eluting fractions 1 to 48, from fractions 36 to 46 were used as a comparative material.

(xi) Formulation of the Samples for the Animal Experiments

To prepare the samples for the animal experiments, aqueous suspensions of the fat-like residues were prepared in commercially available aqua ad injectabilia with the aid of ethyl alcohol (96%). Thus, the fat-like residue was first wetted with the ethanol and filled with the water at a ratio of 1:19 (v/v). This mixture was then homogenized using an ultrasonic microsuspension device once to three times for 15 to 30 seconds each at 5 to 30 Watt output power. As a solvent control, a mixture of ethanol/water (5/95; v/v) was used.

(xii) Dosage of the Mixtures and Mixtures Employed

The above mentioned pooled fractions 1 to 276 in which the hypertensive activity described herein was contained, the pooled fractions 1 to 48 for comparison, also as described above, and the solvent control described were examined on seven rats each. The injected volume of 1.5 ml was injected over about 10 seconds each. The dosage was 10 mg/kg of body weight each.

(xiii) Animal Experiments

For the animal experiments, 21 adult spontaneously hypertensive Wistar rats (SHR/N Crl BR) with an average weight of about 400 g were used. Under Ketanest/diazepam anesthesia (Ketanest 50 mg/kg of body weight; diazepam 3 mg/kg of body weight), the individual doses with a volume of 800 pl were injected within 15 seconds through the vena jugularis into the vena cava superior. The dosage was 50 mg/kg of body weight each, Page 31 of 39 following Lee et al. (Lee, supra). Measurement of the blood pressure was effected intraarterially in the arteria iliaca communis or in the distal aorta, depending on the position of the catheter, from an access on the right arteria femoralis. A commercially available pressure transducer in connection with a Sirecust 404 monitor was used. For calibrating the pressure values to be measured with this unit, a mercury gauge according to Gauer was used. Recording of the blood pressure was performed analogously on a commercially available Kompensograph III recorder as well as digitally through a commercially available personal computer at a sample frequency of 17 Hz. The monitoring period was one hour each.

(xiv) Statistics

For statistical evaluation, the latent period until the maximum of the rise in blood pressure was first established upon administration of the mixture of fractions 1 to 276. Then, the blood pressure values of this time were compared with the blood pressure values obtained immediately prior to the administration of the mixture. Thus, the U test of Whitney and Mann was used with the aid of the program Graph Pad Prism (Graph Pad, U.S.A.). A significant difference was assumed for p <0.05.

(xv) Results of the Animal Experiments

1.) Fractions 1 to 276. In this fraction, a strong hypertensive activity was found upon injection which reached its maximum after 55±27 seconds from the end of the injection and lasted for 201±59 seconds. It can be seen that the blood pressure begins to increase continuously almost immediately after the beginning of the injection, reaches a maximum and then decreases again somewhat more slowly to reach the original level. The rise was 30.1±7.1 mm Hg systolic and 34.7±6 mm Hg diastolic (p=0.0003).
2.) Fractions 1 to 48 (comparative fraction). Injection of this fraction caused no significant change in blood pressure. An essential influence on blood pressure cannot be observed. At the time of the blood pressure maximum after administration of fractions 1 to 276, i.e., 55 seconds after the end of the injection, a slight decrease in blood pressure by 1.1±3 mm Hg systolic and a slight decrease by 0.3±5.1 mm Hg diastolic was recorded. None of these differences was statistically significant.
3.) Solvent control: Upon injection of a mixture of ethanol/water as used for suspending the above described fractions, a significant change in blood pressure could not be found either in these rats. At the time of the blood pressure maximum after administration of fractions 1 to 276, i.e., 55 seconds after the end of the injection, a slight decrease in blood pressure by 0.1±3 mm Hg systolic and a slight decrease by 0.9±2.2 mm Hg diastolic was recorded. None of these differences was statistically significant.

Such an activity as herein described was not found either when other suspensions of fatty fractions were injected.

1. A method for isolating a hypotensive activity from the renal medulla of mammals by
(i) drying the mammal renal medulla;
(ii) extracting the dried renal medulla with an organic solvent having a dielectric constant of between 2 and 45;
(iii) subjecting the extract obtained to chromatography over a hydrophilic sorbent using an eluent gradient of from non-polar to polar; and
(iv) recovering the hypotensive activity as an extract after separating off the eluent.

2. The method according to claim 1, characterized in that said mammals are selected from the group consisting of pigs, rabbits, rats, sheep, dogs, bovines and primates.

3. The method according to claim 1, characterized in that said organic solvent is selected from a C₁-C₃ chlorohydrocarbon or supercritical carbon dioxide.

4. The method according to claim 3, characterized in that said C₁-C₃ chlorohydrocarbon is selected from the group consisting of chloroform, methylene dichloride, 1,1-dichloroethane, 1,2-dichloroethylene and 1,1,2-trichloroethane.

5. The method according to claim 1, characterized in that said hydrophilic sorbent is selected from the group consisting of regular and irregular silica gels.

6. The method according to claim 5, characterized in that said silica gel has a grain size of between 3 and 500 μm and a pore size of between 4 and 12 nm (40 to 120 Å).

7. The method according to claim 1, characterized in that the eluent gradient used in said chromatography consists of C₅-Clo alkane, C₅-C₁₀ alkane/C₂-C₈ ether/ethanol, C₂-C₈ ether/ethanol, C₂-C₈ ether/ethanol/methanol, ethanol/methanol, and methanol.

8. The method according to claim 5, characterized in that said C₅-C₁₀ alkane is selected from the group consisting of n-pentane, n-hexane, cyclohexane, n-heptane, n- or iso-octane, n- or iso-nonane, n- and iso-decane.

9. The method according to claim 8, characterized in that said C₂-C₈ ether is selected from the group consisting of diethyl ether, diisopropyl ether, ethyl isopropyl ether, tert-butyl methyl ether, tetrahydrofuran and dioxan.

10. A hypotensive extract obtainable by the method as defined in any of claims 1 to 9.

11. A pharmaceutical composition comprising the hypotensive extract according to claim 10.

12. Use of the hypotensive extract according to claim 10 or of the pharmaceutical composition according to claim 11 for lowering the blood pressure in mammals.

13. Use of the hypotensive extract according to claim 10 or of the pharmaceutical composition according to claim 11 for preparing a medicament for lowering the blood pressure in mammals. 14. A method for isolating a hypertensive activity from the renal medulla of mammals by
(i) drying the mammal renal medulla;
(ii) extracting the dried renal medulla with an organic solvent having a dielectric constant of between 2 and 45;
(iii) subjecting the extract obtained to chromatography over a hydrophilic sorbent using an eluent gradient of from non-polar to polar;
(iv) obtaining a residue after separating off the eluent; and
(v) recovering the hypertensive activity by subjecting the residue to gel chromatography with an organic solvent having a dielectric constant of between 2 and 45.

15. The method according to claim 14, characterized in that said mammals are selected from the group consisting of pigs, rabbits, rats, sheep, dogs, bovines and primates.

16. The method according to claim 14, characterized in that said organic solvent in step
(ii) is selected from a C₁-C₃ chlorohydrocarbon or supercritical carbon dioxide.

17. The method according to claim 16, characterized in that said C₁-C₃ chlorohydrocarbon is selected from the group consisting of chloroform, methylene dichloride, 1,1-dichloroethane, 1,2-dichloroethylene and 1,1,2-trichloroethane.

18. The method according to claim 14, characterized in that said hydrophilic sorbent is selected from the group consisting of regular and irregular silica gels.

19. The method according to claim 18, characterized in that said silica gel has a grain size of between 3 and 500 μm and a pore size of between 4 and 12 nm (40 to 120 Å).

20. The method according to claim 14, characterized in that the eluent gradient used in said chromatography consists of C₅-C₁₀ alkane, C₅-C₁₀ alkane/C₂-C₈ ether/ethanol, C₂-C₈ ether/ethanol, C₂₋C₈ ether/ethanol/methanol, ethanol/methanol, and methanol.

21. The method according to claim 20, characterized in that said C₅-C₁₀ alkane is selected from the group consisting of n-pentane, n-hexane, cyclohexane, n-heptane, n- or iso-octane, n- or iso-nonane, n- and iso-decane.

22. The method according to claim 20, characterized in that said C₂-C₈ ether is selected from the group consisting of diethyl ether, diisopropyl ether, ethyl isopropyl ether, tert-butyl methyl ether, tetrahydrofuran and dioxan.

23. The method according to claim 14, characterized in that said organic solvent used in the gel chromatography in step (v) is selected from the group consisting of C₁-C₄ alcohols, C₂-C₈ ethers, C₅-C₁₀ aliphatic alkanes, C₆-C₁₂ aromatic hydrocarbons, C₃-C₉, ketones and their mixtures.

24. A hypertensive extract obtainable by the method as defined in any of claims 14 to 23.

25. A pharmaceutical composition comprising the hypertensive extract according to claim 24.

26. Use of the hypertensive extract according to claim 24 or of the pharmaceutical composition according to claim 25 for lowering the blood pressure in mammals.

27. Use of the hypotensive extract according to claim 24 or of the pharmaceutical composition according to claim 25 for preparing a medicament for raising the blood pressure in mammals.
